(19) United States
(12) Patent Application Publication
(10) Pub. No.: US 2010/0273733 A1
(43) Pub. Date: Oct. 28, 2010

(54) HIV Treatment

(76) Inventor: Ruben G. Fabunan, Los Angeles, CA (US)

Correspondence Address: MURPHY DESMOND S.C., P.O. BOX 2038, MADISON, WI, 53701-2038, US

(21) Appl. No.: 12/755,558

(22) Filed: Apr. 7, 2010

Related U.S. Application Data
(60) Provisional application No. 61/173,302, filed on Apr. 28, 2009.

(51) Int. Cl. A61K 31/7072; A61K 31/573; A61P 31/18

(52) U.S. Cl. 514/50; 514/171

(57) ABSTRACT

The invention is directed to a method of treating an HIV infection comprising administering an aminoalkanol and a glucocorticoid. The invention is also directed to a pharmaceutical composition comprising the aminoalkanol and the glucocorticoid in amounts effective to selectively eliminate HIV-infected cells and/or to inhibit HIV replication. A method of enhancing the anti-HIV effect of the antiretroviral drug AZT by administering with the AZT, a pharmaceutical composition comprising an aminoalkanol and a glucocorticoid.

### RELATED APPLICATION

The present invention claims priority from U.S. Provisional Patent Application 61/173,302 filed 28 Apr. 2009.

### BACKGROUND OF THE INVENTION

The present invention provides a treatment to cure or ameliorate the symptoms of HIV infection.

### SUMMARY OF THE INVENTION

The invention is directed to a method of treating an HIV infection comprising administering an aminoalkanol and a glucocorticoid. The invention is also directed to a pharmaceutical composition comprising the aminoalkanol and the glucocorticoid in amounts effective to selectively eliminate HIV-infected cells and/or to inhibit HIV replication. A method of enhancing the anti-HIV effect of the antiretroviral drug AZT by administering with the AZT, a pharmaceutical composition comprising an aminoalkanol and a glucocorticoid.

### DETAILED DESCRIPTION OF THE INVENTION

HIV infection is treated by the administration of a composition comprising an aminoalkanol and a glucocorticoid. Examples of aminoalkanols include, but are not limited to, ethanolamine, methylaminoethanol, ethylaminoethanol, propylaminothanol, butylaminoethanol, isopropylaminoethanol, diisopropylaminoethanol, dipropylaminoethanol, diethylaminoethanol, dimethylethanolamine, propanolamine, diisopropylaminopropanol, dipropylaminopropanol, diethylaminopropanol, dimethylaminopropanol, structural variants and therapeutically effective structural analogs thereof. Examples of glucocorticoids include, but are not limited to, dexamethasone, flumethasone, betamethasone, and structural variants and therapeutically effective structural analogs thereof. More particular examples of the pharmaceutical compositions in accordance with this invention include compositions of diethylaminoethanol (DEAE) and dexamethasone (Dex). In the context of the present disclosure, the named ingredients also include therapeutically effective salts and hydrates, thereof. Therapeutically effective ingredients may be administered as prodrugs such as pharmaceutically acceptable esters of the aminoalkanols or the glucocorticoids.

The term “prodrug,” as used herein, represents compounds which are rapidly biotransformed in vivo to the parent compound by, for example, hydrolysis in the blood. The term “pharmaceutically acceptable ester,” as used herein, represents esters which hydrolyze in vivo and include those that break down readily in the human body to leave the parent compound or a salt thereof. Suitable ester groups include, but are not limited to, those derived from pharmaceutically acceptable aliphatic carboxylic acids, particularly alkanoic, alkenoic, cycloalkanoic and alkanedioic acids, in which each alkyl or alkenyl group preferably has not more than 6 carbon atoms. Other acceptable carboxylates can also include benzoic acids, and derivatives thereof. Examples of particular esters includes formates, acetates, propionates, butyates, acrylates and ethylsuccinates.

The term “therapeutically effective salts or hydrates,” as used herein, represents those salts or hydrates which are, within the scope of sound medical judgment, suitable for use in contact with the tissues of humans and lower animals without undue toxicity, irritation, allergic response and the like and correspond to a reasonable benefit/risk ratio. Pharmaceutically acceptable salts are well-known in the art. The salts can be prepared in-situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

Pharmaceutically effective compositions of this invention may be administered to humans and other animals by a variety of methods that may include continuous or intermittent administration. Examples of methods of administration may include, but are not limited to, oral, rectal, parenteral, intracisternal, intrasternal, intravaginal, intraperitoneal, topical, transdermal, buccal, or as an oral or nasal spray. Accordingly, the pharmaceutically effective compositions herein may also include pharmaceutically acceptable additives, carriers or excipients. Such pharmaceutical compositions may also include the active ingredients formulated together with one or more non-toxic, pharmaceutically acceptable carriers specially formulated for oral administration in solid or liquid form, for parenteral injection or for rectal administration according to standard methods known in the art.

The term “parenteral” administration as used herein, refers to modes of administration which include intravenous, intramuscular, intraperitoneal, intracisternal, intrasternal, subcutaneous and intraarticular injection and infusion. Injectable mixtures are known in the art and comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity may be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents which delay absorption such as aluminum monostearate and gelatin.

Such injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

In some cases, in order to prolong the effect of the drug, it is desirable to slow drug absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, absorption of a parenterally administered drug form may be delayed by dissolving or suspending the drug in an oil vehicle.

Injectable depot forms are made by forming microencapsule matrices of the drugs in biodegradable polymers such as polylactide-polyglycolide. Depending upon the ratio of drugs to polymer and the nature of the particular polymer employed, the rate of drug release can be controlled. Examples of other biodegradable polymers include poly(orthoesters) and poly(anhydrides). Depot injectable formulations are also prepared by entrapping the drugs in liposomes or microemulsions which are compatible with body tissues.

In preparing pharmaceutical compositions in oral dosage form, any of the usual pharmaceutical media known in the art may be used. Solid dosage forms for oral administration include capsules, dragees, tablets, pills, powders and granules. In solid dosage forms, the active compounds are mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner for sustained release by standard techniques. Examples of embedding compositions which can be used include polymeric substances and waxes. The active compounds can also be in micro-encapsulated fowl, if appropriate, with one or more of the above-mentioned excipients.

Liquid dosage forms for oral administration include pharmaceutically acceptable emulsions, solutions, suspensions, syrups and elixirs known in the art. In addition to the active compounds, the liquid dosage foams may contain inert diluents commonly used in the art such as, for example, water, glycols, oils, alcohols or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants and additives such as preservatives wetting agents, emulsifying and suspending agents, coloring, sweetening, flavoring and perfuming agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are known in the art and may include suppositories which can be prepared by mixing the compounds of this invention with suitable non-irritating excipients or carriers such as starch, sugar carriers, such as dextrose, mannitol, lactose, cocoa butter, polyethylene glycol or a suppository wax which are solid at room temperature but liquid at body temperature and therefore melt in the rectum or vaginal cavity and release the active compound. Additional ingredients include diluents, granulating agents, lubricants, binders, disintegrating agents and the like.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art.

Dosage forms for topical administration of a compound of this invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

To prepare the pharmaceutical compositions according to the present invention, an effective amount of the compounds according to the present invention is intimately admixed with a pharmaceutically acceptable carrier according to conventional pharmaceutical compounding techniques to produce a dose. A carrier may take a wide variety of forms depending on the form of preparation desired for administration, e.g., oral or parenteral.

Actual dosage levels of active ingredients in the pharmaceutical compositions of this invention may be varied so as to obtain amounts of the active compounds which are effective to achieve the desired therapeutic response for a particular patient, compositions and mode of administration. The selected dosage level will depend upon the activity of the compounds, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

In an embodiment of the invention used to treat HIV infections in humans, the compositions preferably will comprise about 50 micrograms up to about 650 mg of each aminoalkanol and glucocorticoid, and will be administered in oral dosage form from about one to eight (preferably one to four) times a day. In another embodiment, the compositions are preferably administered parenterally.

Compositions according to the present invention may also be administered in combination with other agents to enhance the biological activity of such agents. Such agents may include any one or more of the standard anti-HIV agents which are known in the art, including, but not limited to, azidothymidine (AZT), dideoxycytidine (ddC), and dideoxyinosine (ddI). Additional agents which have shown anti-HIV effects and may be combined with compositions in accordance to the invention include, for example, raltegravir, maraviroc, bestatin, human chorionic gonadotropin (hCG), levamisole, estrogen, efavirenz, etravirine, indomethacin, emtricitabine, tenofovir disoproxil fumarate, amprenavir, tipranavir, indinavir, ritonavir, darunavir, enfuvirtide, and gramicidin.

Some anticipated modes and doses of administrations are described as follows. In one embodiment, an HIV infected adult human patient is injected intramuscularly with a 2 ml dose of a 9:1 mixture of DEAE (10 mg/ml) and Dex (4 mg/ml) mixture followed by a second dose 90 minutes later. The treatment may be repeated five to seven days (each with a 90 minute interval). Alternatively, this aggressive treatment may be continued twice a day for 15-16 days, followed by administration 5 days a week, 4 days a week, 3 days a week, 2 days a week, and thereafter, once a week for at least one year. In another embodiment, an HIV infected infant patient is injected intramuscularly with a 1 ml dose of a mixture of DEAE and Dex mixture followed by a second dose 120 minutes later. The treatment may be repeated five to seven days or in accordance with any of the extended treatment described above.

In a further embodiment, a patient is administered with an oral dose of 40 mg DEAE and 0.8 mg Dex in solid form until the symptoms of HIV infection is ameliorated. In another embodiment, it is anticipated that a patient will be administered with an oral dose of 30 mg DEAE and 0.6 mg Dex in liquid form twice a day for a week, followed by administration for 4 days in a week, 3 days in a week, and two days in a week. Alternatively, the oral liquid dosage may be administered twice a day for 15-16 days, followed by administration 5 days a week, 4 days a week, 3 days a week, 2 days a week, and thereafter, once a week for at least one year.

More generally, adult HIV patients are treated with between about 1-10 ml intramuscular injection of a mixture comprising between about 0.5-9.5 ml of about 0.5-13% aminoalkanol and between about 0.1-7.5 ml of about 4 mg/ml glucocorticoid. In another embodiment, adult HIV patients are treated with between about 0.1-10 ml intramuscular injection of a mixture having between about 0.1-9.9 ml of about 0.5-11% dimethylaminoethanol and between about 0.1-7.2 ml of about 2-10 mg/ml betamethasone. A further embodiment treats adult HIV patients with between about 1-10 ml intravenous injection of between about 0.5-9.5 ml of about 0.5-30% propylaminopropanol and between about 0.1-5.5 ml of about 2-10 mg/ml Dex.

Yet another embodiment provides between about 0.5-200 mg of DEAE and between about 0.2-80.0 mg Dex in a solid or liquid oral dosage form to an adult HIV patient. In yet another embodiment, an adult HIV patient is treated with between about 0.5-18% DEAE and between about 4 mg/ml Dex in a transdermal patch. In yet another embodiment, an adult HIV patient is treated with between about 0.5-15% diisopropylamino-butanol and between about 4 mg/ml betamethasone in a suppository.

Children are treated with about half the dose for adults. HIV patients may require an extended treatment regimen further comprising additional treatments wherein a composition in accordance to the invention is administered as often as four times a day to as little as once a week.

### EXPERIMENTAL RESULTS

For purposes of the following experiments and unless otherwise specified, the standard DEAE/Dex concentration is about 6.0 to about 6.9 molar DEAE/0.1 molar Dex.

Selective Toxicity. Wells with 10⁶ Jurkat (T-lymphocyte) cells were infected with HIV at Multiplicities of Infection (MOI—the number of viral particles vs. infectable cells) of 0.1 or 0.01 for 4 hours. The cells are treated with Dex, DEAE, DEAE/DEX or control for 48 hours. Cell viability was assayed by MTT or XTT method. In the control (uninfected) cells, standard concentrations of Dex, DEAE and DEAE/Dex are non-toxic (within standard deviation). However, for infected cells, while Dex and DEAE alone did not indicate significant toxicity, the cells that were treated with DEAE/Dex indicated a decrease in viability of between about 30-35%. Other experiments show that HIV alone decreased cell viability to about 85%, but DEAE/Dex decreased cell viability to about 50%.

In another experiment, T-cells were exposed to HIV alone or in combination with drugs. Live cells were measured. No significant toxicity to uninfected cells were found for Dex/DEAE. For cells infected with HIV and treated with Dex/DEAE no viable cells were found (100% treatment). The ability to selectively eliminate infected T-cells without affecting uninfected T-cells is considered a substantial step in the treatment of HIV.

It was expected that with prolonged exposure to treatment with DEAE/Dex, the killing of infected cells would lead to a decline in viral load. This was confirmed using a set of wells from the selective toxicity protocol, and simply sustaining the HIV+ cells exposed to treatments for up to one week by addition of fresh culture media with the same treatment concentrations. This showed that DEAE/Dex gave significant reductions in “pseudo” viral load (PVL), as assessed in TZM-bl cells. After just one week, supernatants of the HIV+ cells treated with DEAE/Dex showed a 42% reduction in relative PVL as compared to untreated HIV+ cells (P<0.01).

AZT Effect. Azidothymidine (AZT) is a well-known HIV treatment. To study the effect of DEAE/Dex on HIV replication, 15,000 TZM-BL (HeLa) cells are plated in each well of 96-well plates. HIV replication is assayed by luciferase response (increased luciferase response indicates increased HIV replication) 48 hours after treatment with the appropriate composition and HIV infection (MOI=0.1). About 40% inhibition of HIV replication was shown by 0.02 μmolar AZT alone and the standard DEAE/Dex composition alone. However, over 60% inhibition was shown by combining the AZT with the DEAE/Dex combination. This appears to support an additive or synergistic enhancement of AZT HIV inhibition by DEAE/Dex.

In another experiment, JC53 cells (HeLa cancer cells engineered to express CD4 and CCR5 receptors for HIV) are treated with HIV and the relevant agents to determine if DEAE inhibit HIV replication, using commercial antibody kit for viral core antigen P24 to assess level of virus production. It was determined that DEAE alone lacked antiviral activity. The virus appears able to propagate and infect other cells. Dex alone indicated a 50% HIV activity inhibition from greater or equal to 0.1 μmolar. But the effect topped out and does not increase with increased concentration. A composition of 0.1 μmolar Dex with 6.0 μmolar DEAE shows about same as Dex alone. Similarly, 0.02 μmolar AZT alone has 40% inhibition of HIV replication. Significantly, AZT with Dex/DEAE indicated 62% inhibition.

Finally, all references, including any priority documents, cited herein are hereby incorporated by reference. While the present invention has been described in considerable detail, it will be obvious to those skilled in the art that alterations may be made in the invention itself or in the procedure for using the invention without departing from the concept and scope of the present invention as described in the following claims.

I claim:

1. A method of selectively eliminating HIV-infected cells in a patient comprising administering to the patient a pharmaceutical composition comprising therapeutically effective amounts of diethylaminoethanol and dexamethasone in a pharmaceutically acceptable carrier.

2. The method according to claim 1, wherein the composition is administered orally.

3. The method according to claim 1, wherein the composition is administered intraveneously.

4. The method according to claim 1, wherein the composition is administered intramuscularly.

5. The method according to claim 1, wherein the composition is administered transdermally.

6. The method according to claim 1, wherein the composition comprises about 0.5 to 200 mg DEAE and between about 0.2-80.0 mg dexamethasone.

7. A method of enhancing the anti-HIV effects of AZT comprising administering to a patient therapeutically effective amounts of diethylaminoethanol, dexamethasone and AZT.

8. The method according to claim 7, wherein the diethylaminoethanol, dexamethasone and AZT are formulated in a single composition.

9. The method according to claim 8, wherein the composition is administered orally.

10. The method according to claim 8, wherein the composition is administered intramuscularly.

11. A method of treating an HIV infected patient comprising administering to the patient therapeutically effective amounts of an aminoalkanol and a glucocorticoid.

12. The method according to claim 11, wherein the aminoalkanol and the glucocorticoid are formulated into a single composition.

13. The method according to claim 12, wherein the composition is administered orally.

14. The method according to claim 11 having between about 0.5 to 200 mg aminoalkanol and between about 0.2-80.0 mg glucocorticoid.

15. The method according to claim 11, wherein the glucocorticoid is selected from the group consisting of dexamethasone, flumethasone and betamethasone.

16. The method according to claim 11, wherein the aminoalkanol is selected from the group consisting of ethanolamine, methylaminoethanol, ethylaminoethanol, propylaminothanol, butylaminoethanol, isopropylaminoethanol, diisopropylaminoethanol, dipropylaminoethanol, diethylaminoethanol, dimethylethanolamine, propanolamine, diisopropylaminopropanol, dipropylaminopropanol, diethylaminopropanol, and dimethylaminopropanol.

17. The method according to claim 11, wherein the glucocorticoid is dexamethasone and the aminoalkanol is diethylaminoethanol.

18. The method according to claim 11, wherein the aminoalkanol is administered in the form of a prodrug.

19. The method according to claim 11, wherein the aminoalkanol and the glucocorticoid are administered in the form of a salt or hydrate.

20. The method according to claim 11, wherein the aminoalkanol and the glucocorticoid are co-administered with an anti-HIV agent selected from the group consisting of AZT, dideoxycytidine, dideoxyinosine, raltegravir, maraviroc, bestatin, hCG, levamisole, estrogen, efavirenz, etravirine, indomethacin, emtricitabine, tenofovir disoproxil fumarate, amprenavir, tipranavir, indinavir, ritonavir, darunavir, enfuvirtide, and gramicidin.
